(19) United States
(12) Patent Application Publication
(10) Pub. No.: US 2011/0129874 A1
(43) Pub. Date: Jun. 2, 2011

(54) Pichia Pastoris Das Promoter Variants

(75) Inventors: Noriko Tsutsumi, Ichikawa-sh (JP); Shinobu Takagi, Funabashi (JP)

(73) Assignee: Novozymes A/S, Bagsvaerd (DK)

(21) Appl. No.: 13/002,631

(22) PCT Filed: Jul. 10, 2009
(86) PCT No.: PCT/EP2009/058859

§ 371 Date: Jan. 4, 2011

Related U.S. Application Data
(60) Provisional application No. 61/080,549, filed on Jul. 14, 2008.

(30) Foreign Application Priority Data
Jul. 11, 2008 (EP) 08160226.0

(51) Int. Cl. C12P 21/02; C07H 21/04; C12N 15/63; C12N 1/19

(52) U.S. Cl. 435/69.1; 536/24.1; 435/320.1; 435/254.23

(57) ABSTRACT

The present invention relates to promoter variants in the form of an isolated polynucleotide comprising: i) a nucleotide sequence consisting of the DAS promoter sequence from Pichia or a functional part thereof, wherein the said DAS promoter is contained in SEQ ID NO: 1; and ii) at least one additional UAS, wherein the said UAS is comprised in SEQ ID NO: 2.

### REFERENCE TO SEQUENCE LISTING

This application contains a Sequence Listing in computer readable form. The computer readable form is incorporated herein by reference.

### FIELD OF THE INVENTION

The present invention relates to an isolated polynucleotide comprising a Pichia DAS promoter variant, a DNA construct comprising the promoter variant operably linked to a polynucleotide encoding a polypeptide of interest, an expression vector comprising the DNA construct, an host cell comprising the DNA construct or the expression vector, a method of producing a polypeptide of interest, a promoter comprising an UAS, and to a use of an UAS for increasing transcription.

### BACKGROUND OF THE INVENTION

Eukaryotic organisms are widely used in industry as host cells for producing polypeptides for, e.g., pharmaceutical and industrial applications. The ability to manipulate gene transcription and expression gives the basis for providing higher production yields.

Conventionally, maximal expression of a gene in a eukaryotic organism is achieved by amplifying in the chromosome an expression cassette containing a single promoter operably linked to a gene encoding the polypeptide of interest and an amplifier selective marker.

Controlled expression is often desirable. In methylotrophic yeast it has been known for long that certain promoters are dependent on the presence of methanol in the growth medium for the induction of transcription. This induction by methanol requires the presence of additional factors, however, the exact mechanism of action for such factors have not been elucidated. Examples of positive factors known from yeast include Mxr1 p, described as a key positive regulator required for methanol utilization in Pichia pastoris (Lin-Cereghino et al., 2006, Mol Cell Biol 26(3): 883-897).

Examples of these methanol dependent promoters have been described in several yeast cells belonging to the group of yeast known as methylotrophic yeast. The promoters controlling expression of the enzymes involved in methanol metabolism in these organisms are particularly strong, and these promoters are generally used to control the heterologous expression of proteins in yeast. However, the specific carbon source used for the cultivation of these host cells has an enormous influence on the regulation of methanol metabolism promoters. Methanol and glycerol are considered as adequate substrates for methylotrophic yeast expression systems, while glucose has been considered inadequate (EP 299108). It is therefore desirable if expression from the known methanol metabolism promoters can be made less dependent on the substrate.

### SUMMARY OF THE INVENTION

The invention provides improved variants of the Pichia DAS promoter for increased expression of a polypeptide of interest.

In a first aspect the present invention relates to an isolated polynucleotide comprising: i) a nucleotide sequence consisting of the DAS promoter sequence from Pichia or a functional part thereof, wherein the said DAS promoter is comprised in SEQ ID NO: 1; and ii) at least one additional UAS, wherein the said UAS is comprised in SEQ ID NO: 2.

In a second aspect the invention relates to a DNA construct comprising a polynucleotide sequence of the invention (modified DAS promoter) operably linked to a structural gene encoding a polypeptide of interest and a terminator.

In a third aspect the invention relates to an expression vector comprising a DNA construct of the invention, further comprising a signal peptide coding region.

In a fourth aspect the present invention relates to a Pichia host cell comprising an expression vector of the invention.

In a fifth aspect the invention relates to a method of producing a polypeptide of interest comprising:

(a) cultivating the host cell of the invention, under conditions conducive for the production of the polypeptide of interest; and

(b) recovering the polypeptide.

In a sixth aspect the invention relates to a promoter comprising an UAS selected from the group consisting of:

i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or

ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and
   wherein the UAS according to (i) or (ii) is either foreign to the promoter or present in more than one copy.

In a seventh aspect the invention relates to a use of an UAS for increasing transcription from a promoter, wherein the UAS is selected from the group consisting of:

i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or

ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and
   wherein the UAS according to (i) or (ii) is either foreign to the promoter or present in more than one copy.

### BRIEF DESCRIPTION OF DRAWINGS

FIG. 1 shows a deletion analysis of the DAS promoter shown in SEQ ID NO: 1.

FIG. 2 shows the position of DAS promoter variants having internal deletions and the numbers of the applied primers.

FIG. 3 shows DAS promoter variants according to the invention having multiple UASs. The DNA sequence of the pDAS wt2 as shown in the figure from the NsiI site and including the region encoding the N-terminal of the phytase corresponds to SEQ ID NO: 6.

FIG. 4 shows further deletion variants of the DAS promoter having internal deletions around the −255 position.

### DEFINITIONS

Identity: For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Hybridization: For purposes of the present invention, hybridization indicates that the nucleotide sequence hybridizes to a labeled nucleic acid probe corresponding to sequence in question e.g. SEQ ID NO: 7; its full-length complementary strand; or a subsequence thereof; under very low to very high stringency conditions. Molecules to which the nucleic acid probe hybridizes under these conditions can be detected using, for example, X-ray film.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at 45° C. (very low stringency), more preferably at 50° C. (low stringency), more preferably at 55° C. (medium stringency), more preferably at 60° C. (medium-high stringency), even more preferably at 65° C. (high stringency), and most preferably at 70° C. (very high stringency).

For short probes that are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes that are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

Subsequence: The term “subsequence” is defined herein as a nucleotide sequence having one or more (several) nucleotides deleted from the 5′ and/or 3′ end of the sequence of SEQ ID NO: 1; wherein the subsequence has promoter activity (thus a functional part thereof). In a preferred aspect, a subsequence contains at least 755 nucleotides, more preferably at least 555 nucleotides, even more preferably at least 455 nucleotides, and most preferably at least 355 nucleotides of the sequence of SEQ ID NO: 1 corresponding to positions 301-1055, 501-1055, 601-1055 and 701-1055 of SEQ ID NO: 1 respectively.
Isolated polynucleotide: The term “isolated polynucleotide” as used herein refers to a polynucleotide that is isolated from a source. In a preferred aspect, the polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by agarose electrophoresis.
Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotides of the present invention are preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.
cDNA: The term “cDNA” is defined herein as a DNA molecule that can be prepared by reverse transcription from a mature, spliced, mRNA molecule obtained from a eukaryotic cell. cDNA lacks intron sequences that are usually present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.
Nucleic acid construct: The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.
Control sequences: The term “control sequences” is defined herein to include all components necessary for the expression of a polynucleotide encoding a polypeptide of the present invention. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.
Operably linked: The term “operably linked” denotes herein a configuration in which a control sequence is placed at an appropriate position relative to the coding sequence of the polynucleotide sequence such that the control sequence directs the expression of the coding sequence of a polypeptide.
Expression: The term “expression” includes any step involved in the production of the polypeptide including, but not limited to, transcription, post-transcriptional modification, translation, post-translational modification, and secretion.
Expression vector: The term “expression vector” is defined herein as a linear or circular DNA molecule that comprises a polynucleotide encoding a polypeptide of the present invention and is operably linked to additional nucleotides that provide for its expression.
Host cell: The term “host cell”, as used herein, includes any cell type that is susceptible to transformation, transfection, transduction, and the like with a nucleic acid construct or expression vector comprising a polynucleotide of the present invention.
Foreign: The term “foreign” means herein that the upstream activating sequence (UAS) according to the invention is derived from a different origin, where “origin” may refer to the gene or cell. Thus for example the UAS is normally found in the promoter region of the Pichia pastoris DAS promoter, however, it may according to the invention be used in a different promoter that naturally does not contain the UAS. Thus the UAS may derive from different genes from the same cell or it may derive from functionally equivalent genes from genetically different cells/species.

### DETAILED DESCRIPTION OF THE INVENTION

The present invention relates to the controlled expression of polypeptides from methanol inducible promoters. Examples of these promoters have been described in several yeast cells belonging to the group of yeast known as methylotrophic yeast. In the context of the present invention a methylotrophic yeast is defined as a group of yeast which can utilize methanol as a sole carbon source for their growth. The promoters for the enzymes involved in methanol metabolism in these organisms are particularly strong, and these promoters (methanol metabolism promoters) are generally used to control the heterologous expression of proteins in yeast.

Known members of methylotrophic yeast host cells belong to the genera selected from the group consisting of Pichia, Hansenula, Candida, Torulopsis. According to the invention the Pichia host cell can in one embodiment be selected from the group consisting of P. pastoris, P. methanolica, P. angusta, P. thermomethanolica. The Hansenula or Candida host cells can be selected from the group consisting of H. polymorpha, and C. boidinii.

Several promoters have previously been isolated and described in the literature from which the expression of heterologous polypeptides can be controlled by the addition of methanol to the growth medium. Such promoters include but are not limited to e.g. the AOX1 promoter (Alcohol Oxidase promoter), DHAS promoter (or DAS promoter) (dihydroxyacetone synthase promoter), FDH promoter (or FMDH promoter) (formate dehydrogenase promoter), MOX promoter (Methanol Oxidase promoter), AOX2 promoter, ZZA1, PEX5-, PEX8-, PEX14-promoter. Particularly the promoter relevant for the present invention is the dihydroxyacetone synthase (DAS or DHAS) promoter.

Normally all of the above promoters require the presence of methanol for their induction. This induction by methanol requires the presence of additional factors (such as transcription factors), however, the exact mechanism of action for such factors have not been elucidated. In yeast e.g. Mxr1p, has been described as a key positive regulator required for methanol utilization in Pichia pastoris (Lin-Cereghino et al., 2006, Mol Cell Biol 26(3): 883-897).

The inventors of the present invention have previously discovered that the controlled expression of a single positive factor, encoded by the Prm1 gene from Pichia pastoris, as described elsewhere (co-pending application WO 2008/090211; priority date 26 Jan. 2007), can be sufficient in order to induce transcription from several methanol inducible promoters without the need for methanol in the growth medium. This was demonstrated using the Prm1 protein as a model protein for the positive activator and using the AOX1 or the DAS promoters for the controlled expression of a reporter polypeptide. The results obtained have shown that it is possible to induce the AOX1 or the DAS promoters simply by controlling the expression of the prm1 gene and without the presence of methanol in the growth medium.

The mechanism of action of the positive regulator, Prm1, has not been elucidated but one possibility is that the regulator binds to the promoter region of the methanol inducible promoter. The inventors of the present invention have identified one such region within the DNA sequence comprising the DAS promoter from Pichia pastoris that could possibly contain a binding region for Prm1.

As described elsewhere (WO 2008/090211/PCT/EP2008/050870) a fragment comprising the DAS promoter from Pichia pastoris can be obtained on a 1055 bp fragment (SEQ ID NO: 1).

In order to test promoter activity for analysis of modified promoter variants the promoter needs to be operably linked to a reporter gene. Any gene the expression of which can be easily determined may be used. A suitable reporter gene may be the Citrobacter braakii phytase gene which has been codon optimized for expression in Pichia fused in frame to the alpha factor signal peptide from S. cerevisiae. The nucleic acid sequence encoding the signal peptide may also advantageously be codon optimized for Pichia expression. The complete DNA sequence for such a reporter gene construct is shown in SEQ ID NO: 4. (For details on how to construct this reporter gene construct see the co-pending application WO 2008/090211). The alpha factor signal peptide is encoded for in positions 1 to 255 of SEQ ID NO: 4. The reporter gene can then be fused to the fragment (SEQ ID NO: 1) comprising the DAS promoter. One such construct is shown in SEQ ID No: 5 comprising the promoter and the start of the phytase gene. The start codon of the signal peptide can be found in position 1065 in SEQ ID NO: 5.

Using the above construct inserted into an appropriate expression vector (any vector that support expression in Pichia) deletion analyzes were performed. From these analyses, as explained in details in the examples, it can be concluded that the 1055 bp DAS promoter fragment (SEQ ID NO: 1) contains a region which appears to be an Upstream Activating Sequence (UAS). This UAS sequence seems to be contained in a 100 bp fragment (position 701 to 800 in SEQ ID NO: 1). When this fragment is added in 1, 2, or 3 copies to a subsequence of the 1055 bp fragment a significant increase in promoter activity could be observed. The Pichia pastoris DAS promoter is therefore comprised in the 1055 bp fragment and the UAS is comprised in the 100 bp fragment.

In one embodiment the present invention therefore relates to an isolated polynucleotide comprising:

i) a nucleotide sequence consisting of the DAS promoter sequence from Pichia or a functional subsequence thereof, wherein the said DAS promoter is comprised in SEQ ID NO: 1; and

ii) at least one additional UAS, wherein the said UAS is comprised in SEQ ID NO: 2.

From the performed analysis it can be seen that the DAS promoter in another embodiment is comprised in a 855 bp subsequence corresponding to position 201 to 1055 in SEQ ID NO: 1, particularly in a 755 bp subsequence corresponding to position 301 to 1055 in SEQ ID NO: 1, more particularly in a 655 bp subsequence corresponding to position 401 to 1055 in SEQ ID NO: 1, more particularly in a 555 bp subsequence corresponding to position 501 to 1055 in SEQ ID NO: 1, even more particularly in a 455 bp subsequence corresponding to position 601 to 1055 in SEQ ID NO: 1, most particularly in a 355 bp subsequence corresponding to position 701 to 1055 in SEQ ID NO: 1.

By analysing the promoter sequence further 3 possible TATA boxes at positions 882, 955, and 1002 in SEQ ID NO: 1 respectively are revealed. In one embodiment the promoter comprises at least the TATA box at position 882. In another embodiment the promoter comprises at least the TATA box at position 955. In still another embodiment the promoter comprises at least the TATA box at position 1002.

The UAS is comprised in the 100 bp subsequence corresponding to position 701 to 800 in SEQ ID NO: 1. However the UAS may be smaller than 100 bp. Within the 100 bp subsequence approximately 20 bp from position 767 to 788 in SEQ ID NO: 1 appears to be essential for proper function.

In a further embodiment the UAS therefore comprises at least the subsequence from position 767 to 788 in SEQ ID NO: 1.

Adding even further UASs will increase the promoter activity even more. The highest activity was seen with three additional UASs. In one embodiment therefore the promoter according to the invention comprises at least two additional UASs, particularly at least three additional UASs, more particularly at least four additional UASs, and even more particularly at least five additional UASs.

It could be envisioned that the number cannot be increased indefinitely however, in case the number of additional UASs becomes too high expression of the positive activator Prm1 can be increased as well, or alternatively that the Mxr1 positive activator level may be increased.

In one embodiment the modified DAS promoter according to the invention comprises one additional UAS positioned upstream of a 855 bp subsequence of SEQ ID NO: 1. Therefore one embodiment relates to an isolated polynucleotide according to the invention, wherein the promoter is chosen from the group consisting of:

a) a polynucleotide comprising or consisting of position 77 to 828, particularly 77 to 901, more particularly 77 to 948 of SEQ ID NO: 7;

b) a polynucleotide comprising or consisting of a polynucleotide having at least 90% identity, preferably at least 95%, more preferably at least 97%, even more preferably at least 99% identity with position 77 to 828, particularly 77 to 901, more particularly 77 to 948 of SEQ ID NO: 7;

c) a polynucleotide comprising or consisting of polynucleotide that hybridizes under at least high stringency conditions with position 77 to 828, particularly 77 to 901, more particularly 77 to 948 of SEQ ID NO: 7 or a full-length complementary strand thereof.

In another embodiment the modified DAS promoter according to the invention comprises two additional UASs positioned upstream of a 855 bp subsequence of SEQ ID NO: 1. Therefore one embodiment relates to an isolated polynucleotide according to the invention, wherein the promoter is chosen from the group consisting of:

a) a polynucleotide comprising or consisting of position 60 to 920, particularly 60 to 993, more particularly 60 to 1040 of SEQ ID NO: 8;

b) a polynucleotide comprising or consisting of a polynucleotide having at least 90% identity, preferably at least 95%, more preferably at least 97%, even more preferably at least 99% identity with position 60 to 920, particularly 60 to 993, more particularly 60 to 1040 of SEQ ID NO: 8;

c) a polynucleotide comprising or consisting of polynucleotide that hybridizes under at least high stringency conditions with position 60 to 920, particularly 60 to 993, more particularly 60 to 1040 of SEQ ID NO: 8 or a full-length complementary strand thereof.

In another embodiment the modified DAS promoter according to the invention comprises three additional UASs positioned opstream of a 855 bp subsequence of SEQ ID NO: 1. Therefore one embodiment relates to an isolated polynucleotide according to the invention, wherein the promoter is chosen from the group consisting of:

a) a polynucleotide comprising or consisting of position 48 to 1015, particularly 48 to 1088, more particularly 48 to 1135 of SEQ ID NO: 9;

b) a polynucleotide comprising or consisting of a polynucleotide having at least 90% identity, preferably at least 95%, more preferably at least 97%, even more preferably at least 99% identity with position 48 to 1015, particularly 48 to 1088, more particularly 48 to 1135 of SEQ ID NO: 9;

c) a polynucleotide comprising or consisting of polynucleotide that hybridizes under at least high stringency conditions with position 48 to 1015, particularly 48 to 1088, more particularly 48 to 1135 of SEQ ID NO: 9 or a full-length complementary strand thereof.

The modified DAS promoters of the invention will be useful for expression of any polypeptide of interest in Pichia, and e.g. in Hansenula polymorpha and Candida boidinii or other methylotrophic yeast. In another aspect the invention thus relates to a DNA construct comprising a polynucleotide sequence (modified DAS promoter) of the invention operably linked to a structural gene encoding a polypeptide of interest and a terminator.

The modified DAS promoters of the invention may also advantageously be used in any suitable Pichia expression plasmid. The skilled person will know how to clone the promoter into such a construct. In a further embodiment the invention therefore relates to an expression vector comprising a DNA construct of the invention, further comprising a signal peptide coding region.

In a still further aspect the invention relates to a Pichia host cell comprising an expression vector of the invention. Particularly the Pichia host cell is a Pichia pastoris host cell.

In an even further aspect the present invention relates to a method of producing a polypeptide of interest comprising:

(a) cultivating the host cell of the invention, under conditions conducive for the production of the polypeptide of interest; and

(b) recovering the polypeptide.

In the above production method the positive regulator Prm 1 will be produced by the host cell since the prm 1 gene is endogenous to Pichia pastoris. However, overproducing Prm 1 can further increase promoter activity, especially when the UAS is present in multiple copies. Even over-expressing the Mxr1 protein may have an effect on the modified DAS promoter activity.

In one embodiment the invention therefore relates to a method according to the invention for producing a polypeptide of interest, wherein expression of the positive regulator Prm1 is increased in the host cell by controlling the expression of Prm1 or by increasing the copy number of the gene encoding Prm1. In an even further embodiment expression of the positive regulator Mxr1 is increased by controlling the expression of Mxr1 or by increasing the copy number of the gene encoding Mxr1. In one additional embodiment both regulators Prm1 and Mxr1 are expressed at increased levels.

In one embodiment the positive regulator is expressed constitutively from a suitable promoter. Preferably the promoter is not the native promoter meaning that the promoter controlling the expression of the positive regulator is different from the promoter normally controlling the expression. In the context of the present invention such preferred promoters are termed “non-native”. The promoter could still be native to the host organism but it will be foreign in the context of the gene in question, e.g. the prm1 gene. In one particular embodiment the promoter is selected from the group consisting of the GAP promoter (glyceraldehyde-3-phosphate dehydrogenase promoter), the TEF1 promoter (Translational elongation factor EF-1 alpha promoter), and the PGK promoter (phosphoglycerate kinase promoter). The host cell according to the invention would normally express the positive regulator from an endogenous gene present on the chromosome in addition to the expression controlled by the non-native promoter as described above. In a further embodiment the endogenous copy of the gene encoding the positive regulator could be inactivated, e.g. by deletion, or the normal promoter controlling the endogenous copy of the gene could be replaced by the chosen non-native promoter.

In another embodiment the expression of the positive regulator is controlled from an inducible promoter which is not methanol inducible.

As described above the positive regulator according to the invention may also be a functional homologue of Prm1 isolated from other yeast cells. According to one embodiment of the invention one such candidate could be Mut3 encoded by the mut3 gene from Hansenula polymorpha (syn. Pichia angusta). In the examples provided herein Prm1 or Mxr1 have been overproduced in Pichia pastoris. It is however possible that the same effect can be obtained by overproducing Mut3 in Pichia or Prm1 in Hansenula or Mut3 in Hansenula. This has not been tested.

Therefore in a further embodiment of the invention the positive regulator is Mut3.

An increase in the level of positive regulator present in the host cell can also be provided by simply having multiple copies of the gene encoding the regulator present in the host cell.

A further aspect of the invention relates to the UAS comprised in SEQ ID NO: 2 or at least comprising SEQ ID NO: 3.

The present invention therefore relates to an isolated polynucleotide selected from the group consisting of:
(a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof.

In a further embodiment the present invention relates to an isolated polynucleotide selected from the group consisting of:
(a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof.

The UAS according to the invention can be used for activating foreign promoters as well as the DAS promoter. In this case one or more copies of the UAS according to the invention is combined with a promoter that in its natural form does not contain the UAS as explained herein. In a further aspect the present invention therefore relates to a promoter comprising an UAS according to the invention, wherein the UAS is foreign to the promoter or wherein the UAS is present in more than one copy.

In another embodiment the present invention relates to a use of the UAS for increasing transcription from a promoter. The promoter may be a foreign promoter in which case it does not already contain a copy of the UAS or it may be the DAS promoter in which case additional copies can be added as described above.

Thus in a further embodiment the invention relates to a promoter comprising an UAS selected from the group consisting of:

i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or

ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and

wherein the UAS according to (i) or (ii) is either foreign to the promoter or present in more than one copy.

In still another aspect the invention relates to a use of an UAS for increasing transcription from a promoter, wherein the UAS is selected from the group consisting of:

i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or

ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and

wherein the UAS according to (i) or (ii) is either foreign to the promoter or present in more than one copy.

Promoters suitable for the above activation may in particular be promoter induced by methanol.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides produced by the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

In one particular embodiment the polypeptide produced from the host cell is heterologous to the host cell. In another embodiment the polypeptide is homologous to the host cell.

### EXAMPLES

### Materials and Methods

### Strains

E. coli TOP10 (invitorgen) and DH5alpha (TOYOBO) were used for a plasmid construction. LB (1% Tripton (Difco), 0.5% Yeast extract (Difco), 0.5% NaCl) was used as base medium after supplement of relevant antibiotics.
Pichia pastoris his4 mutant, GS115, was used for the expression test.
Pichia pastoris COls702 (Muts) is a AOX1 gene disrupted strain of Pichia pastoris NRRL Y-15851 and is described in example 5.
The used media for its growth are as following:
YPD (2% Pepton (Difco), 1% Yeast extract (Difco), 2% Glucose)
RD Agar; 1M sorbitol, 2% Glucose, 1.34% Yeast nitrogen base (Difco), 4×10⁻⁵% biotin, 0.005% amino acids (L-glutamic acid, L-methionine, L-lysine, L-leucine, and L-isoleucin), 2% Agar Noble (Difco)
MD Agar; 1.34% Yeast nitrogen base, 4×10⁻⁵% Biotin, 2% Glucose

### Transformation of Pichia Pastoris:

Pichia pastoris strains were transformed by electroporation. Fresh competent cells were prepared by the following procedure. The host strain, was inoculated to 100 ml of YPD and grown till OD660 is 1.2˜1.4. The cells were washed with ice-cold water twice (100 ml, and 50 ml), and with 4 ml of ice-cold 1M sorbitol. Then the cells were suspended 0.2 ml of ice-cold 1M sorbitol. Linearized plasmid DNA (1˜2 μg) was mixed with 80 μl of fresh competent cells and stored on ice for 5 min. Cells were transferred to an ice-cold 0.2 cm electroporation cuvette. Transformation was performed using a BioRad™ GenePulser II. Parameters used were 1500 V, 25 μF and 200Ω. Immediately after pulsing, cells are suspended in 1 ml of ice cold 1 M sorbitol. The mixtures were plated on the relevant selection plates.

### Colony PCR for Screening of His4 Locus Integration

The cell was picked with sterilized tooth picks to a 0.2 ml tube then baked in a microwave oven. The dried cell was suspended in 50 μl of sterilized water and it was subjected to PCR using Expand High Fidelity plus (Roche). The reaction mixture was 20 μl including 2 mM dNTP, 10 microM of each primer, 1 unit of Expand high fidelity plus (Roche), 1× Expand high fidelity plus buffer (Roche), and 1 μl of cell suspension mentioned above. The PCR primers were primer139; 5′-ctgctctagccagtttgctg-3′ (SEQ ID NO: 10) (upstream of His4 maker in genome) and S98; 5′-gccgcccagtcctgctcgct-3′ (SEQ ID NO: 11) (between His4 marker and AOX terminator in expression plasmids). The PCR program is as below.

| | | |
|---|---|---|
| temp | time | cycle |

| 94° C. | 2 | min | 1 |
|---|---|---|---|
| 94° C. | 10 | sec | 11 |
| 55° C. | 30 | sec | |
| 68° C. | 3 | min | |
| 94° C. | 10 | sec | 20 |
| 55° C. | 30 | sec | |
| 68° C. | 3 min + 20 sec/cycle | | |
| 68° C. | 7 | min | 1 |

The strains in which the expression cassette was integrated at His4 locus showed about 2.9 kb band.

Shake Flask Evaluation with Methanol Induction

Cell cultivated on the MD agar for 3 days was inoculated in 100 ml of YPD in 500 ml of SF and cultivated at 30° C. with shaking. Then 1 ml of the seed culture was inoculated in 100 ml of YPD and cultivated at 30° C. for 2 days. During cultivation, 5 ml of 40% methanol was added for induction on day 2. Sampling was carried out on day 3.

### Phytase Assay Method:

7.5 mM of sodium phytate dissolved in the acetate buffer pH 5.5 is mixed with ½ volume of enzyme sample solution in the same acetate buffer containing 0.01% Tween 20. After the incubation at 37° C. for 30 mines, the stop reagent containing 20 mM ammonium heptamolybdate and 0.06% ammonium vanadate dissolved in 10.8% nitric acid was added to generate yellow complex with released inorganic phosphate. The amount of released phosphate is measured photometrically as the absorbance at 405 nm. One phytase unit is defined as the amount of enzyme to release 1 μmol inorganic phosphate per minute.

TABLE 1
Primers and primer combinations

| Primer | sequence | direction | plasmid |
|---|---|---|---|
| primer126 | 5′ gaaaagtgccacctgagtccgttactcaaaaagtc 3′ | forward | pDd-2 |
| primer127 | 5′ gaaaagtgccacctgcggtatctgaaacacaagac 3′ | forward | pDd-3 |
| primer128 | 5′ gaaaagtgccacctgtgaaaattactggtaacttc 3′ | forward | pDd-4 |
| primer129 | 5′ gaaaagtgccacctgctataagaaacgaaggattt 3′ | forward | pDd-5 |
| primer130 | 5′ gaaaagtgccacctgaaagaaattcggacaaatag 3′ | forward | pDd-6 |
| primer132 | 5′ gaaaagtgccacctgttctccacattcagtcatag 3′ | forward | pDd-8 |
| primer133 | 5′ gaaaagtgccacctgcagtttaaagtttttctttc 3′ | forward | pDd-9 |
| primer134 | 5′ gaaaagtgccacctgttataacccctctaaacact 3′ | forward | pDd-10 |
| primer135 | 5′ aaccagcccagtgggacgtcccattgtggccact- | reverse | pDd- |
| | gatctgggg 3′ | | 2~20 |
| primer144 | 5′ ccccgaaaagtgccacctgacgtc 3′ | forward | pDd- |
| | | | 11~20 |
| primer152 | 5′ actgaatgtggagaacgatggtgttgctcaa- | reverse | pDd-14, |
| | caaagggatg 3′ | | pDd-20 |
| primer153 | 5′ ttgagcaacaccatcgttctccacattcagtcata- | forward | pDd- |
| | gatggg 3′ | | 14, 20 |
| primer154 | 5′ aacagataatagaaattctccacattcagtcata- | forward | pDd-15 |
| | gatggg 3′ | | |
| primer155 | 5′ actgaatgtggagaatttctattatctgttcgtttgggcg 3′ | reverse | pDd-15 |
| primer156 | 5′ ttagtatacttgcccttctccacattcagtcatagatggg 3′ | forward | pDd-16 |
| primer157 | 5′ actgaatgtggagaagggcaagtatac- | reverse | pDd-16 |
| | taaatctcggaac 3′ | | |
| primer158 | 5′ aacagataatagaaattagccagtacgaaagag- | forward | oDd-17 |
| | gaaactt 3′ | | |
| primer159 | 5′ tttcgtactggctaatttctattatctgttcgtttgggcg 3′ | reverse | pDd-17 |
| primer160 | 5′ ttagtatacttgcccttagccagtacgaaagag- | forward | pDd-18 |
| | gaaactt 3′ | | |
| primer161 | 5′ tttcgtactggctaagggcaagtatac- | reverse | pDd-18 |
| | taaatctcggaac 3′ | | |
| primer162 | 5′ ttagtatacttgcccaaagaaattcggacaaata- | forward | pDd-19, |
| | gaacac 3′ | | 20 |
| primer163 | 5′ tgtccgaatttctttgggcaagtatac- | reverse | pDd-19, |
| | taaatctcggaaca 3′ | | 20 |
| primer174 | 5′ taggctaaact- | reverse | pDd32 |
| | cattcgcgctcggttaagtttcctctttcg 3′ | | |
| primer175 | 5′ gcgcgaatgagtttagcctagatatcctt 3′ | forward | pDd32 |
| primer176 | 5′ aagtatcggaacaactaaactcattcgcgcctta- | reverse | pDd33 |
| | gatttc 3′ | | |
| primer177 | 5′ gtttagttgttccgatacttctccacattc 3′ | forward | pDd33 |
| primer196 | 5′ aagatgcttttctgtgactggtgagtactcaaccaagt- | forward | pDd32, |
| | ca 3′ | | 33 |
| primer201 | 5′ ttccccgaaaagtgccacctgacgtcgttggcgcgcc | forward | pDd26, |
| | aaggactagt 3′ | | 27, 28 |
| primer202 | 5′ agacaagttctccaccacgtggagtcaac- | reverse | pDd26, |
| | cagcccagtgggacgtc 3′ | | 27, 28 |

TABLE 2
Primer combination to make the promoter
variants of pDd2~20, pDd32, and pDd33

| | 1st PCR | 1st PCR | 2nd PCR | 2nd PCR |
|---|---|---|---|---|
| Plasmid | (forward) | (reverse) | (forward) | (reverse) |
| pDd-2 | primer126 | primer135 | | |
| pDd-3 | primer127 | primer135 | | |
| pDd-4 | primer128 | primer135 | | |
| pDd-5 | primer129 | primer135 | | |
| pDd-6 | primer130 | primer135 | | |
| pDd-8 | primer132 | primer135 | | |
| pDd-9 | primer133 | primer135 | | |
| pDd-10 | primer134 | primer135 | | |
| pDd-14 | primer144 | primer152 | primer144 | primer135 |
| | primer153 | primer135 | | |
| pDd-15 | primer144 | primer155 | primer144 | primer135 |
| | primer154 | primer135 | | |
| pDd-16 | primer144 | preimr157 | primer144 | primer135 |
| | primer156 | primer135 | | |
| pDd-17 | primer144 | primer159 | primer144 | primer135 |
| | primer158 | primer135 | | |
| pDd-18 | primer144 | primer161 | primer144 | primer135 |
| | primer160 | primer135 | | |
| pDd-19 | primer144 | primer163 | primer144 | primer135 |
| | primer162 | primer135 | | |
| pDd-20 | primer144 | primer163 | primer144 | primer135 |
| | primer162 | primer152 | | |
| | priemr153 | primer135 | | |
| pDd-32 | primer 196 | primer174 | primer 196 | primer 135 |
| | primer 175 | primer135 | | |
| pDd-33 | primer196 | primer176 | primer 196 | primer 135 |
| | primer 177 | primer135 | | |

### Example 1

### Construction and Expression Evaluation DAS1 Promoter Variants

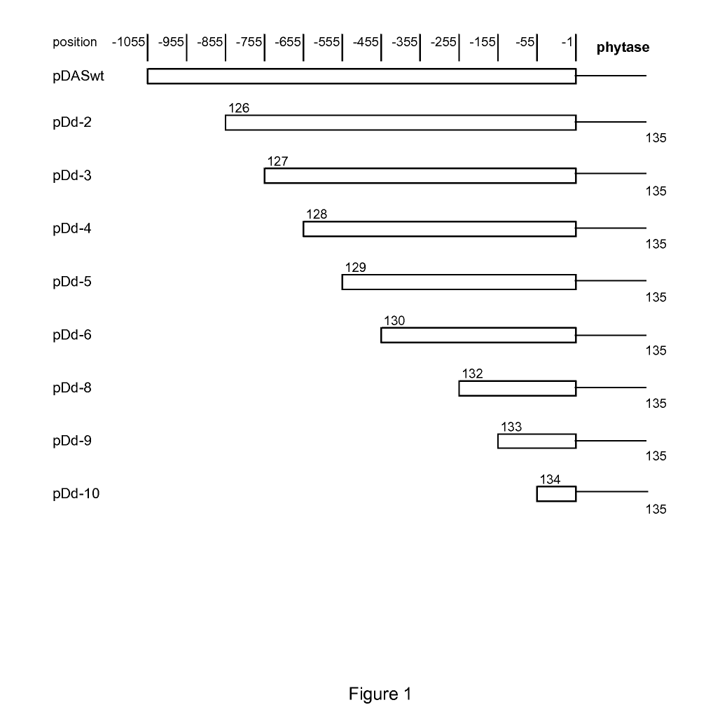

Cloning of DAS promoter of Pichia pastoris has been described previously (WO 2008/090211). An expression cassette consisting of the wt DAS promoter from Pichia pastoris controlling the expression of a phytase gene, optimized for expression in Pichia, was used for the construction of promoter variants (deletion variants). The complete sequence of the promoter fragment is shown in SEQ ID NO: 1, the reporter gene in SEQ ID NO: 4 and the fusion construct (expression cassette) is shown in SEQ ID NO: 5 (DAS wt promoter and 5′-end of the phytase coding sequence including a codon optimized alpha factor signal peptide encoding sequence). Promoter variants of different length (as shown in FIG. 1 in which position −1 corresponds to position 1055 in SEQ ID NO: 1 and position −1055 corresponds to position 1 in SEQ ID NO: 1) were prepared by PCR using the primers shown in Table 1 and the primer combination sets shown in Table 2. The template DNA was the wild type DAS promoter. The expression cassette can be inserted in any appropriate Pichia expression plasmid having an appropriate selection marker. Preferably the selection marker is HIS4. One such construct is pDAS1 wt.

PCR was carried out using the 50 microL of reaction including 2 mM dNTP, 10 microM of each primer, 2.8 unit of Expand high fidelity plus (Roche), 1× Expand high fidelity plus buffer (Roche), and 2 ng of the template plasmid DNA. The PCR program is as below.

| | | |
|---|---|---|
| Temp | time | cycle |

| 94° C. | 2 | min | 1 |
|---|---|---|---|
| 94° C. | 10 | sec | 11 |
| 55° C. | 30 | sec | |
| 68° C. | 3 | min | |
| 94° C. | 10 | sec | 20 |
| 55° C. | 30 | sec | |
| 68° C. | 3 min + 20 sec/cycle | | |
| 68° C. | 7 | min | 1 |

The amplified DNA fragment was purified by gel extraction kit (Qiagen) and used for the construction of phytase expression plasmids.

For making expression plasmids, the amplified fragment was sub-cloned by In-Fusion PCR cloning kit (Clontech) into the template phytase expression vector (pDAS1 wt) digested with the AatII restriction enzyme.

Resulting expression plasmids (pDd-2 through pDd-10) were linearized by digestion with SalI and transformed into Pichia pastoris his4 strain. The strains in which the expression cassette was integrated at the HIS4 locus were screened by colony PCR. The selected strains were cultivated in liquid medium with methanol induction and phytase activity in the culture broth was measured. The results are shown in Table 3.

TABLE 3

| Strains | | Promoter | Relative activity |
|---|---|---|---|
| (series) | plasmid | length (bp) | (%) |
| DASwt | pDAS1 wt | 1050 | 100 |
| 2 | pDd-2 | 854 | 155 |
| 3 | pDd-3 | 754 | 102 |
| 4 | pDd-4 | 654 | 128 |
| 5 | pDd-5 | 554 | 102 |
| 6 | pDd-6 | 454 | 78 |
| 8 | pDd-8 | 254 | 24 |
| 9 | pDd-9 | 154 | 13 |
| 10 | pDd-10 | 54 | 14 |

### Example 2

### Construction and Expression Evaluation DAS1 Promoter Variants (2)

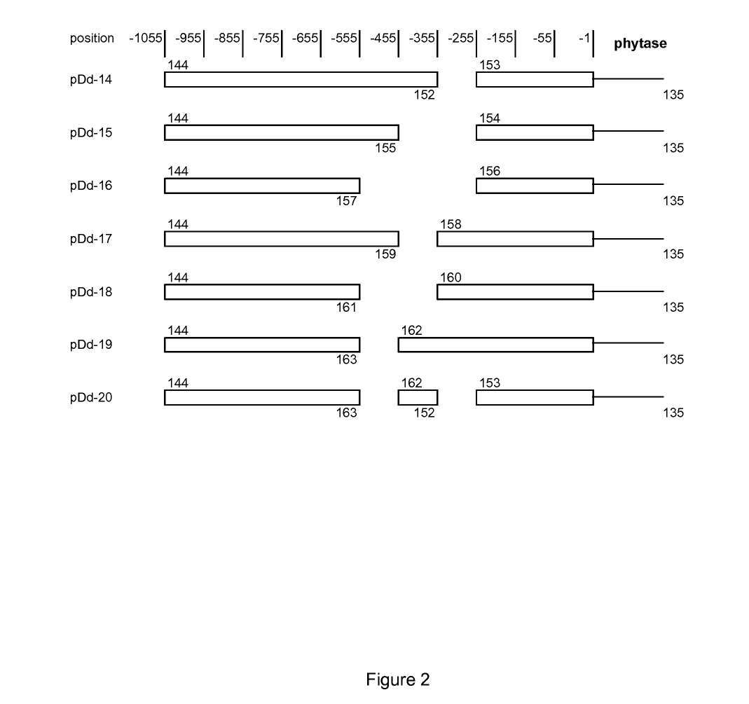

DAS promoter variants which possess internal deletions as shown in FIG. 2 were constructed by SOE PCR. The 1st PCR was carried out using the 50 microL of reaction including 2 mM dNTP, 10 microM of each primer shown in Table 2, 2.8 unit of Expand high fidelity plus (Roche), 1× Expand high fidelity plus buffer (Roche), and 2 ng of the plasmid DNA of pDAS1 wt. The PCR program is as below.

| | | |
|---|---|---|
| temp | time | cycle |

| 94° C. | 2 | min | 1 |
|---|---|---|---|
| 94° C. | 10 | sec | 11 |
| 55° C. | 30 | sec | |
| 68° C. | 3 | min | |
| 94° C. | 10 | sec | 20 |
| 55° C. | 30 | sec | |
| 68° C. | 3 min + 20 sec/cycle | | |
| 68° C. | 7 | min | 1 |

The purified fragments from 1st PCR were subjected to the 2nd PCR using the 50 microL of reaction including 2 mM dNTP, 10 microM of primer 144 and primer135, 2.8 unit of Expand high fidelity plus (Roche), 1× Expand high fidelity plus buffer (Roche), and purified PCR product of the 1^{st} run. The PCR program is as below.

| | | |
|---|---|---|
| Temp | time | cycle |

| 94° C. | 2 | min | 1 |
|---|---|---|---|
| 94° C. | 10 | sec | 11 |
| 60° C. | 30 | sec | |
| 68° C. | 3 | min | |
| 94° C. | 10 | sec | 20 |
| 60° C. | 30 | sec | |
| 68° C. | 3 min + 20 sec/cycle | | |
| 68° C. | 7 | min | 1 |

Structure of each promoter variants and the used primers are shown in FIG. 2. The sequence corresponding to the pDAS1 wt fragment shown in FIG. 1 is shown in SEQ ID NO: 1 (promoter) and SEQ ID NO: 5 (promoter+alpha leader+N-terminal phytase). The phytase expression plasmids carrying the DAS1 promoter variants (pDd-14 through pDd20) were generated, and transformants of Pichia pastoris were isolated as previously shown. The selected strains were cultivated in YPD medium with methanol induction. The phytase activity in the culture broth was measured and results were shown in Table 4.

TABLE 4

| Strains | | | Relative activity |
|---|---|---|---|
| (series) | plasmid | Deleted region | (%) |
| DASwt | pDAS1 wt | no | 100 |
| 14 | pDd-14 | −355~−255 | 5 |
| 16 | pDd-16 | −555~−255 | 2 |
| 17 | pDd-17 | −455~−355 | 68 |
| 18 | pDd-18 | −555~−355 | 75 |
| 19 | pDd-19 | −555~−455 | 77 |
| 20 | pDd-20 | −555~−455 | 2 |
| | | −355~−255 | |

It was found that the deletion between −355 and −255 (containing in pDd-14, 16, 20) drastically reduced the expression level. Therefore, it was concluded that the region is an Upstream Activation Region (UAS) of DAS1 wt promoter.

### Example 3

### Amplification of UAS in DAS Promoter

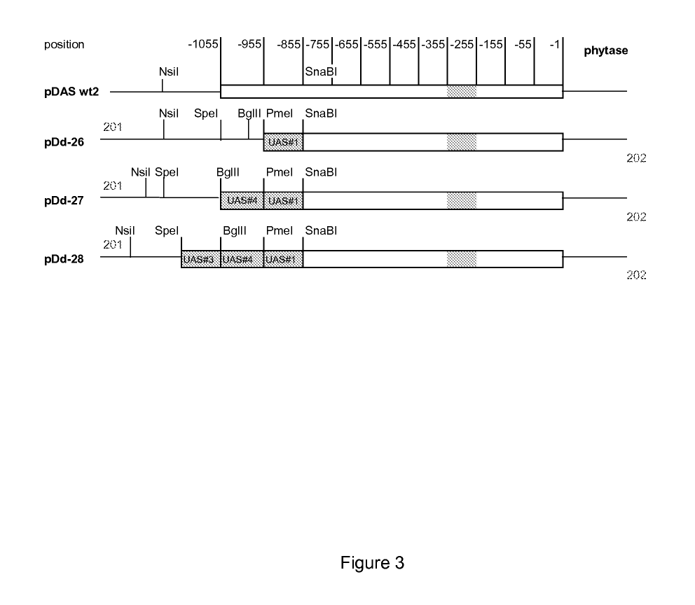

DAS promoter variants in which the number of the UAS is amplified were constructed. The resulting constructs are illustrated in FIG. 3. The UAS was amplified by PCR and the constructs were based on the wild type DAS promoter shown as pDAS wt2 in the figure and the complete sequence from the NsiI site and including the phytase gene encoding the N-terminal is shown in SEQ ID NO: 6. The amplification resulted in constructs having multiple copies of the UAS as shown in FIG. 3 and by using the primers 201 and 202 these constructs were sub-cloned into pDAS1 wt.

The promoter region and a part of phytase gene were amplified by PCR using primer 201 and primer 202 with the 50 microL of reaction mixture including 2 mM dNTP, 10 microM of each primer, 2.8 unit of Expand high fidelity plus (Rosche), 1× Expand high fidelity plus buffer (Rosche), and 2 ng of the plasmid of template DNA. The PCR program is as below.

| | | |
|---|---|---|
| temp | time | cycle |

| 94° C. | 2 | min | 1 |
|---|---|---|---|
| 94° C. | 10 | sec | 11 |
| 55° C. | 30 | sec | |
| 68° C. | 3 | min | |
| 94° C. | 10 | sec | 20 |
| 55° C. | 30 | sec | |
| 68° C. | 3 min + 20 sec/cycle | | |
| 68° C. | 7 | min | 1 |

The amplified DNA fragment was purified and sub-cloned into pDAS1 wt digested with PmII and AatII using the In-fusion cloning kit. The amplification resulted in pDd-26, pDd-27, and pDd-28 and the complete sequences corresponding to the region marked by the primers 201 and 202 are shown in SEQ ID NO: 7, 8, and 9 respectively. These promoter cassettes can then be used in an appropriate expression plasmid of choice.

Each expression plasmid was integrated at HIS4 locus of Pichia pastoris, and the expression level with methanol induction was evaluated in YPD medium. The results are shown in Table 5.

TABLE 5

| Strains | | Number of | Relative activity |
|---|---|---|---|
| (series) | plasmid | additional UAS | (%) |
| 2 | pDd-2 | 0 | 100 |
| 26 | pDd-26 | 1 | 108 |
| 27 | pDd-27 | 2 | 139 |
| 28 | pDd-28 | 3 | 181 |

It can be seen from the results that increasing the number of the UAS will result in an increase in promoter activity.

### Example 4

### Construction and Expression Evaluation DAS1 Promoter Variants (3)

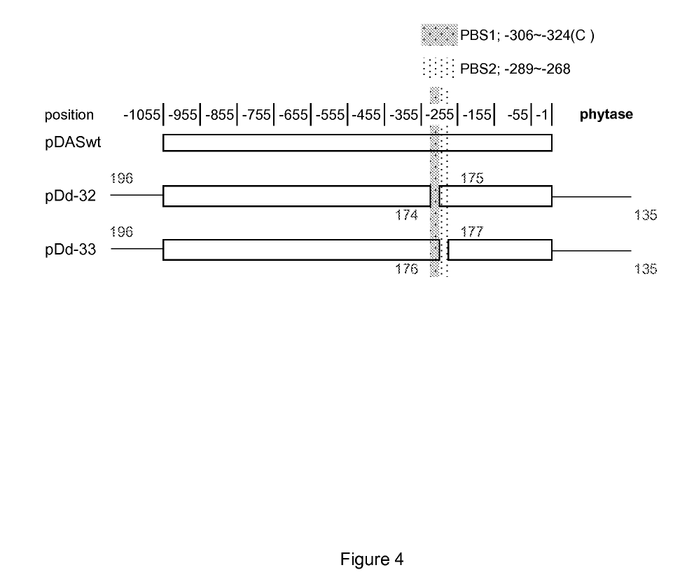

In order to narrow down the essential region in the UAS, further deletion variants were created (FIG. 4). Two region, −306 to −324 (PBS1) and −289 to −268 (PBS2), corresponding to position 732-750 and 767-788 in SEQ ID NO: 1 respectively, were predicted as potential binding sites of transcription factor, and variants having these regions deleted were constructed by SOE-PCR as described in Example 2. The amplified DNA fragment was purified by gel extraction kit (Qiagen) and used for the construction of expression plasmid of phytase.

For making expression plasmids, the amplified fragment was sub-cloned by In-Fusion PCR cloning kit (Clontech) into the template phytase expression vector pDAS1 wt digested by the SacI and SnaBI restriction sites. The primers are shown in Table 1 and 2.

Each expression plasmid was integrated at HIS4 locus of Pichia pastoris, and the expression level with methanol induction was evaluated in YPD medium. The results are shown in Table 6.

TABLE 6
The result of SF evaluation (pDd-32 and pDd-33)

| Strains | | | Relative activity |
|---|---|---|---|
| (series) | plasmid | deletion | (%) |
| DP3 | pNo-DP3 | no | 100 |
| 32 | pDd-32 | PBS1 | 75 |
| 33 | pDd-33 | PBS2 | 24 |

### Example 5

### Expression of Humicula Insolens Cutinase from Modified DAS Promoter with Prm1 and/or Mxr1 Over-Expression

The aim of this experiment was to check whether the expression of a protein of interest, exemplified by the Humicola insolence cutinase gene controlled by an improved DAS promoter according to the invention, having four repeats of the UAS, could be improved by co-expression of transcription factors, such as Prm1 and/or Mxr1 in Pichia pastoris. The host strain used was COls702 (Mut^{s}).

COLs702 was constructed from Pichia pastoris NRRL Y-15851, which has a mutation in the his4 gene to make the gene inactive. NRRL Y-15851 was transformed with pCOls693 (SEQ ID NO: 41) in standard manner. An aox1 deleted strain, COLs702, was obtained using a traditional approach, by transformation with the marker gene, SUC2, flanked by locus specific deletion fragments. The plasmid pCOls693 has a SUC2 gene from S. cerevisiae as the marker gene and flanking sequences from the aox1 gene. Transformants were isolated using sucrose as the sole carbon source. Due to the his4 negative genotype of the mother strain, histidine was supplemented to the selection agar medium. Fast growing transformants on the selection plate were isolated.

Isolated transformants were studied by PCR to confirm the aimed insertion of SUC2 gene into AOX1 locus. Resulting strains have the AOX1 gene disrupted by this event. One of the strains was named as Pichia pastoris COLs702 for further use.

A re-transformation of low-expressing Humicola insolens cutinase transformants comprising pNori58-HIC (wt Humicula insolens cutinase controlled by the improved DAS promoter), pNori58-RSII0014 (H. insolens cutinase variant controlled by improved DAS promoter), or pNori58-RSII0007 (H. insolens cutinase variant controlled by improved DAS promoter), was carried out with the plasmids harboring Prm1 (pGPrm, SEQ ID NO: 42) or Mxr1 (pGMxr, SEQ ID NO: 43) or both, and their expression was analyzed. Both plasmids have the Zeocin resistant gene as a selection marker gene and the regulator gene is controlled by the GAP promoter. A homologous recombination event at the GAP promoter region of the respective vectors pNori58 and pGPrm/pGMxr was expected. pGPrm comprises the expression cassette (SEQ ID NO: 44) having the GAP promoter in position 1-483, Prm1 CDS in position 490-3549, terminator in position 3487-3827. pGMxr comprises the expression cassette (SEQ ID NO: 45) having the GAP promoter in position 1-483, Mxr1 CDS in position 493-3957, terminator in position 4028-4368.

For transformations, plasmid DNA (pGMxr and pGPrm) was linearized with AvrII and cleaned-up using Biorad clean-up kit.

After heat shock at 42° C., the cells were harvested and re-suspended in 1 ml YPDS w/o Zeocin and incubated with shaking at 30° C., 200 rpm for 3 h. The cells were then harvested, resuspended in 100 μl of the supernatant and plated on YPDS plates containing 100 μg/ml Zeocin.

One transformant from each of the above transformations that showed expression in small scale was chosen for expression in large scale: inoculation in shake flasks with 150 ml Buffered Sorbitol Complex media (BSCM) in 500 ml baffled flasks. After ˜24 h of growth, the cultures were induced with 100% methanol to a final concentration of 1%. Once induction was started, the cotton plugs were replaced with four layers of gauze cloth+1 layer of Cheese cloth. Induction was carried out every 24 h at 22° C., 150 rpm for 3 days with induction and without induction for 2.5 days (over the weekend). An aliquot of culture (500 μl) was pelleted by centrifugation at 13000 rpm for 5 min and the supernatants (12 μl) analyzed for protein expression on a 12% SDS-PAGE with coomassie stain.

From the gel-data the following expression levels could be seen:

| Enzyme expression plasmid | Prm1/Mxr1 plasmid | Expression level |
|---|---|---|
| pNori58-HIC | none | +++ |
| | pGPrm | +++ |
| | pGMxr/pGPrm | +++ |
| pNori58-RSII0007 | None | — |
| | pGMxr | + |
| | pGMxr/pGPrm | — |
| pNori58-RSII0014 | None | ++ |
| | pGMxr | +++ |
| | pGPrm | ++ |
| | pGMxr/pGPrm | +++ |

The obtained data indicate an improved expression of the test protein when Mxr protein was over-expressed.

1. An isolated polynucleotide comprising:
i) a nucleotide sequence consisting of the DAS promoter sequence from Pichia or a functional part thereof, wherein the said DAS promoter is comprised in SEQ ID NO: 1; and
ii) at least one additional UAS, wherein the said UAS is comprised in SEQ ID NO: 2.

2. The isolated polynucleotide according to claim 1, wherein the UAS comprises at least the sequence shown in SEQ ID NO: 3.

3. The isolated polynucleotide according to claim 1, wherein the DAS promoter is comprised in position 501 to 1055 in SEQ ID NO: 1.

4. The isolated polynucleotide according to claim 1, wherein the DAS promoter is comprised in position 601 to 1055 in SEQ ID NO: 1.

5. The isolated polynucleotide according to claim 1, comprising at least two additional UASs.

6. The isolated polynucleotide according to claim 1, comprising at least three additional UASs.

7. The isolated polynucleotide according to claim 1, wherein the promoter is chosen from the group consisting of:
a) a polynucleotide comprising or consisting of position 77 to 828, particularly 77 to 901, more particularly 77 to 948 of SEQ ID NO: 7;
b) a polynucleotide comprising or consisting of a polynucleotide having at least 90% identity, preferably at least 95%, more preferably at least 97%, even more preferably at least 99% identity with position 77 to 828, particularly 77 to 901, more particularly 77 to 948 of SEQ ID NO: 7;
c) a polynucleotide comprising or consisting of polynucleotide that hybridizes under at least high stringency conditions with position 77 to 828, particularly 77 to 901, more particularly 77 to 948 of SEQ ID NO: 7 or a full-length complementary strand thereof.

8. The isolated polynucleotide according to claim 1, wherein the promoter is chosen from the group consisting of:
a) a polynucleotide comprising or consisting of position 60 to 920, particularly 60 to 993, more particularly 60 to 1040 of SEQ ID NO: 8;
b) a polynucleotide comprising or consisting of a polynucleotide having at least 90% identity, preferably at least 95%, more preferably at least 97%, even more preferably at least 99% identity with position 60 to 920, particularly 60 to 993, more particularly 60 to 1040 of SEQ ID NO: 8;
c) a polynucleotide comprising or consisting of polynucleotide that hybridizes under at least high stringency conditions with position 60 to 920, particularly 60 to 993, more particularly 60 to 1040 of SEQ ID NO: 8 or a full-length complementary strand thereof.

9. The isolated polynucleotide according to claim 1, wherein the promoter is chosen from the group consisting of:
a) a polynucleotide comprising or consisting of position 48 to 1015, particularly 48 to 1088, more particularly 48 to 1135 of SEQ ID NO: 9;
b) a polynucleotide comprising or consisting of a polynucleotide having at least 90% identity, preferably at least 95%, more preferably at least 97%, even more preferably at least 99% identity with position 48 to 1015, particularly 48 to 1088, more particularly 48 to 1135 of SEQ ID NO: 9;
c) a polynucleotide comprising or consisting of polynucleotide that hybridizes under at least high stringency conditions with position 48 to 1015, particularly 48 to 1088, more particularly 48 to 1135 of SEQ ID NO: 9 or a full-length complementary strand thereof.

10. A DNA construct comprising a polynucleotide sequence of claim 1 operably linked to a structural gene encoding a polypeptide of interest and a terminator.

11. An expression vector comprising a DNA construct of claim 10, further comprising a signal peptide coding region.

12. A Pichia host cell comprising an expression vector of claim 10.

13. A method of producing a polypeptide of interest comprising:
(a) cultivating the host cell of claim 12, under conditions conducive for the production of the polypeptide of interest; and
(b) recovering the polypeptide.

14. The method according to claim 13, wherein expression of the positive regulator Prm1 is increased by controlling the expression of Prm1 or by increasing the copy number of the gene encoding Prm1.

15. The method according to claim 13, wherein expression of the positive regulator Mxr1 is increased by controlling the expression of Mxr1 or by increasing the copy number of the gene encoding Mxr1.

16. The method according to claim 13, wherein expression of the positive regulators Prm1 and Mxr1 are increase at the same by controlling their expression or by increasing the copy number of the respective genes.

17. A promoter comprising an UAS selected from the group consisting of:
i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or
ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and
wherein the UAS according to (i) or (ii) is either foreign to the promoter or present in more than one copy.

18. A use of an UAS for increasing transcription from a promoter, wherein the UAS is selected from the group consisting of:
i) (a) a polynucleotide comprising or consisting of SEQ ID NO: 2; or
(b) a polynucleotide comprising or consisting of a polynucleotide having at least 90% identity, preferably at least 95%, more preferably at least 97%, even more preferably at least 99% identity with SEQ ID NO: 2; or
c) a polynucleotide comprising or consisting of polynucleotide that hybridizes under at least high stringency conditions with SEQ ID NO: 2 or a full-length complementary strand thereof; or
ii) (a) a polynucleotide comprising or consisting of SEQ ID NO: 3; or
(b) a polynucleotide comprising or consisting of a polynucleotide having at least 90% identity, preferably at least 95%, more preferably at least 97%, even more preferably at least 99% identity with SEQ ID NO: 3; or
c) a polynucleotide comprising or consisting of polynucleotide that hybridizes under at least high stringency conditions with SEQ ID NO: 3 or a full-length complementary strand thereof; and
wherein the UAS according to (i) or (ii) is either foreign to the promoter or present in more than one copy.

19. The use according to claim 18, wherein the promoter is induced by methanol.


SEQUENCE LISTING (45 sequences)

SEQ ID NO: 1 - DNA, 1055 bp, Pichia pastoris
atagggagaaaaaccgagacaacgatggaactcccatgtagattccaccgccccaattactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 2 - DNA, 100 bp, Pichia pastoris
ttagccagtacgaaagaggaaacttaaccgataccttggagaaatctaaggcgcgaatgagtttagcctagatatccttagtgaagggttgttccgatac

SEQ ID NO: 3 - DNA, 22 bp, Pichia pastoris
cctagatatccttagtgaaggg

SEQ ID NO: 4 - DNA, 1491 bp, Artificial Sequence
  alpha factor signal peptide fused to Citrobacter braakii phytase both optimized for expression in Pichia
atgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtctctgaattgggtcagtaccagagactgtggttcacctccaaaggacttctgaataaccaaacttgcccatccccaggacaagtcgctgttattgccgacaccgatcaaagaaccagaaaaaccggagaggcctttttggcaggacttgctccaaaatgccagattcaagtccactaccaaaaagacgaagagaagaacgatccattgttcaatcccgtcaagatgggaaaatgctcctttaacaccttgcaagtcaaaaacgccattttggaaagagcaggtggcaatatcgagctttacacccagcgttaccaatcttcttttagaactttggaaaatgttttgaactttagtcagtccgagacttgcaagaccaccgagaagtctaccaagtgcactttgcccgaggctttgccctccgagcttaaggtcactcccgataacgtctccttgccaggagcatggtctctttcctccactttgaccgagattttcttgttgcaggaggcacaaggaatgccacaggtcgcatggggtagaattaccggtgaaaaggaatggagagacttgctgtctcttcacaacgcccagttcgatctcttgcagagaaccccagaggttgccagatccagagctactccacttttggatatgatcgacaccgctttgctgaccaatggtaccaccgagaacagatacggtattaagttgccagtctccttgctgttcattgcaggtcacgacaccaatttggccaacttgtctggagccttggacctgaactggtctttgccaggacagcccgacaataccccaccaggaggcgaattggttttcgaaaagtggaaaagaacctccgataacaccgattgggtccaagtctccttcgtctaccaaaccttgagagatatgcgtgacattcagccactgtctttggagaagcccgctggtaaggttgacttgaaattgatcgcttgcgaagaaaagaactcccagggaatgtgctctttgaagtccttttccagattgatcaaggagattagagtccccgagtgcgctgtcaccgagtaa

SEQ ID NO: 5 - DNA, 1485 bp, Artificial Sequence
  DAS promoter fused to phytase
gacgtcatagggagaaaaaccgagacaacgatggaactcccatgtagattccaccgccccaattactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatatgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtct

SEQ ID NO: 6 - DNA, 1493 bp, Artificial Sequence
  DAS promoter fused to phytase
tcatgcatgacgtcatagggagaaaaaccgagacaacgatggaactcccatgtagattccaccgccccaattactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatatgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtct

SEQ ID NO: 7 - DNA, 1421 bp, Artificial Sequence
  Promoter variant fused to phytase reporter gene
ttccccgaaaagtgccacctgacgtcgttggcgcgccaaggactagtcccggaagatcttccagctttgtttaaacttagccagtacgaaagaggaaacttaaccgataccttggagaaatctaaggcgcgaatgagtttagcctagatatccttagtgaagggttgttccgatactacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatatgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtct

SEQ ID NO: 8 - DNA, 1513 bp, Artificial Sequence
  Promoter variant fused to phytase reporter gene
ttccccgaaaagtgccacctgacgtcgttggcgcgccaaggactagtcccggaagatctttagccagtacgaaagaggaaacttaaccgataccttggagaaatctaaggcgcgaatgagtttagcctagatatccttagtgaagggttgttccgatacggtttaaacttagccagtacgaaagaggaaacttaaccgataccttggagaaatctaaggcgcgaatgagtttagcctagatatccttagtgaagggttgttccgatactacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatatgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtct

SEQ ID NO: 9 - DNA, 1608 bp, Artificial Sequence
  Promoter variant fused to phytase reporter gene
ttccccgaaaagtgccacctgacgtcgttggcgcgccaaggactagtttagccagtacgaaagaggaaacttaaccgataccttggagaaatctaaggcgcgaatgagtttagcctagatatccttagtgaagggttgttccgatacgagatctttagccagtacgaaagaggaaacttaaccgataccttggagaaatctaaggcgcgaatgagtttagcctagatatccttagtgaagggttgttccgatacggtttaaacttagccagtacgaaagaggaaacttaaccgataccttggagaaatctaaggcgcgaatgagtttagcctagatatccttagtgaagggttgttccgatactacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatatgagattcccatccatcttcactgctgttttgttcgctgcttcctccgctttggctgctccagttaacactactactgaagatgaaactgctcaaatcccagctgaagctgttatcggttactccgacttggaaggtgatttcgacgttgctgttttgccattctccaactccaccaacaacggattgttgttcattaacaccaccattgcttccatcgctgctaaggaagaaggtgtttccttggaaaagagagaggaacagaatggaatgaagttggagagagttgtcatcgtttctagacacggtgttagagctcccaccaaattcactccaatcatgaagaacgtcaccccagatcagtggccacaatgggacgtcccactgggctggttgactccacgtggtggagaacttgtct

SEQ ID NO: 10 - DNA, 20 bp, Artificial Sequence
  PCR primer
ctgctctagccagtttgctg

SEQ ID NO: 11 - DNA, 20 bp, Artificial Sequence
  PCR primer
gccgcccagtcctgctcgct

SEQ ID NO: 12 - DNA, 35 bp, Artificial Sequence
  PCR primer
gaaaagtgccacctgagtccgttactcaaaaagtc

SEQ ID NO: 13 - DNA, 35 bp, Artificial Sequence
  PCR primer
gaaaagtgccacctgcggtatctgaaacacaagac

SEQ ID NO: 14 - DNA, 35 bp, Artificial Sequence
  PCR primer
gaaaagtgccacctgtgaaaattactggtaacttc

SEQ ID NO: 15 - DNA, 35 bp, Artificial Sequence
  PCR primer
gaaaagtgccacctgctataagaaacgaaggattt

SEQ ID NO: 16 - DNA, 35 bp, Artificial Sequence
  PCR primer
gaaaagtgccacctgaaagaaattcggacaaatag

SEQ ID NO: 17 - DNA, 35 bp, Artificial Sequence
  PCR primer
gaaaagtgccacctgttctccacattcagtcatag

SEQ ID NO: 18 - DNA, 35 bp, Artificial Sequence
  PCR primer
gaaaagtgccacctgcagtttaaagtttttctttc

SEQ ID NO: 19 - DNA, 35 bp, Artificial Sequence
  PCR primer
gaaaagtgccacctgttataacccctctaaacact

SEQ ID NO: 20 - DNA, 43 bp, Artificial Sequence
  PCR primer
aaccagcccagtgggacgtcccattgtggccactgatctgggg

SEQ ID NO: 21 - DNA, 24 bp, Artificial Sequence
  PCR primer
ccccgaaaagtgccacctgacgtc

SEQ ID NO: 22 - DNA, 41 bp, Artificial Sequence
  PCR primer
actgaatgtggagaacgatggtgttgctcaacaaagggatg

SEQ ID NO: 23 - DNA, 41 bp, Artificial Sequence
  PCR primer
ttgagcaacaccatcgttctccacattcagtcatagatggg

SEQ ID NO: 24 - DNA, 40 bp, Artificial Sequence
  PCR primer
aacagataatagaaattctccacattcagtcatagatggg

SEQ ID NO: 25 - DNA, 40 bp, Artificial Sequence
  PCR primer
actgaatgtggagaatttctattatctgttcgtttgggcg

SEQ ID NO: 26 - DNA, 40 bp, Artificial Sequence
  PCR primer
ttagtatacttgcccttctccacattcagtcatagatggg

SEQ ID NO: 27 - DNA, 40 bp, Artificial Sequence
  PCR primer
actgaatgtggagaagggcaagtatactaaatctcggaac

SEQ ID NO: 28 - DNA, 40 bp, Artificial Sequence
  PCR primer
aacagataatagaaattagccagtacgaaagaggaaactt

SEQ ID NO: 29 - DNA, 40 bp, Artificial Sequence
  PCR primer
tttcgtactggctaatttctattatctgttcgtttgggcg

SEQ ID NO: 30 - DNA, 40 bp, Artificial Sequence
  PCR primer
ttagtatacttgcccttagccagtacgaaagaggaaactt

SEQ ID NO: 31 - DNA, 40 bp, Artificial Sequence
  PCR primer
tttcgtactggctaagggcaagtatactaaatctcggaac

SEQ ID NO: 32 - DNA, 40 bp, Artificial Sequence
  PCR primer
ttagtatacttgcccaaagaaattcggacaaatagaacac

SEQ ID NO: 33 - DNA, 41 bp, Artificial Sequence
  PCR primer
tgtccgaatttctttgggcaagtatactaaatctcggaaca

SEQ ID NO: 34 - DNA, 41 bp, Artificial Sequence
  PCR primer
taggctaaactcattcgcgctcggttaagtttcctctttcg

SEQ ID NO: 35 - DNA, 29 bp, Artificial Sequence
  PCR primer
gcgcgaatgagtttagcctagatatcctt

SEQ ID NO: 36 - DNA, 40 bp, Artificial Sequence
  PCR primer
aagtatcggaacaactaaactcattcgcgccttagatttc

SEQ ID NO: 37 - DNA, 30 bp, Artificial Sequence
  PCR primer
gtttagttgttccgatacttctccacattc

SEQ ID NO: 38 - DNA, 40 bp, Artificial Sequence
  PCR primer
aagatgcttttctgtgactggtgagtactcaaccaagtca

SEQ ID NO: 39 - DNA, 47 bp, Artificial Sequence
  PCR primer
ttccccgaaaagtgccacctgacgtcgttggcgcgccaaggactagt

SEQ ID NO: 40 - DNA, 46 bp, Artificial Sequence
  PCR primer
agacaagttctccaccacgtggagtcaaccagcccagtgggacgtc

SEQ ID NO: 41 - DNA, 9667 bp, Artificial Sequence
  complete plasmid sequence
ccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgcaagcttcaacgatgccaaaagggtataataagcgtcatttgcagcattgtgaagaaaactatgtggcaagccaagcctgcgaagaatgtattttaagtttgactttgatgtattcacttgattaagccataattctcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccagacgataataccaatctaaactctttaaacgttaaaggacaagtatgtctgcctgtattaaaccccaaatcagctcgtagtctgatcctcatcaacttgaggggcactatcttgttttagagaaatttgcggagatgcagatcttaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgcccttagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatggctatccccgaagagtttgatatccccctgtagtaaacagggagataccgtacggaggtctgaattccctacagaagtagctgtaaaaattcagaattcgcaacaacctataattgagttaagtgcctttccaagctaaaaagtttgaggttataggggcttagcatccacacgtcacaatctcgggtatcgagtatagtatgtagaattacggcaggaggtttcccaatgaacaaaggacaggggcacggtgagctgtcgaaggtatccattttatcatgtttcgtttgtacaagcacgacatactaagacatttaccgtatgggagttgttgtcctagcgtagttctcgctcccccagcaaagctcaaaaaagtacgtcatttagaatagtttgtgagcaaattaccagtcggtatgctacgttagaaaggcccacagtattcttctaccaaaggcgtgcctttgttgaactcgatccattatgagggcttccattattccccgcatttttattactctgaacaggaataaaaagaaaaaacccagtttaggaaattatccgggggcgaagaaatacgcgtagcgttaatcgaccccacgtccagggtttttccatggaggtttctggaaaaactgacgaggaatgtgattataaatccctttatgtgatgtctaagacttttaaggtacgcccgatgtttgcctattaccatcatagagacgtttcttttcgaggaatgcttaaacgactttgtttgacaaaaatgttgcctaagggctctatagtaaaccatttggaagaaagatttgacgactttttttttttggatttcgatcctataatccttcctcctgaaaagaaacatataaatagatatgtattattcttcaaaacattctcttgttcttgtgctttttttttaccatatatcttacttttttttttctctcagagaaacaagcaaaacaaaaagcttttcttttcactaacgtatatgatgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatagaggttataaaacttattgtcttttttatttttttcaaaagccattctaaagggctttagctaacgagtgacgaatgtaaaactttatgatttcaaagaatacctccaaaccattgaaaatgtatttttatttttattttctcccgaccccagttacctggaatttgttctttatgtactttatataagtataattctcttaaaaatttttactactttgcaatagacatcattttttcacgtaataaacccacaatcgtaatgtagttgccttacactactaggatggacctttttgcctttatctgttttgttactgacacaatgaaaccgggtaaagtattagttatgtgaaaatttaaaagcattaagtagaagtataccatattgtaaaaaaaaaaagcgttgtcttctacgtaaaagtgttctcaaaaagaagtagtgagggaaatggataccaagctatctgtaacaggagctaaaaaatctcagggaaaagcttctggtttgggaaacgaaggaactccaatcgggaatgaggagagcacaaataaagctaaaaatggaaataaaaagcggaataaaaatcgaaacagaaataagaagacagagacaaaggaacaaaatgaacctaaacctgtgactggaggggaagaagtgagagtcgagaaatctcaagcgaaaaacagaaggcggaaaaacaataatggagctaataaaaagaataccctacactactcaaaggaaattaatgtagaagagcgaaaacaaatcgcaaaaaggcaagaagaaatcgaacaatgcattcacaccttatccgattttaaactgtttaaaaagggtaagcacgttacctcatatggctatcgaatctcccccatgacagactccggtaagatttctttgaaaattttattcaacatacctttggattatccaaaggccccaataaaactaacgatgaaaagtaatgaagaagtttcctcttacatggacaccgccc

SEQ ID NO: 42 - DNA, 5741 bp, Artificial Sequence
  complete plasmid sequence
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgaaatgcctcctaaacatcggctggagcagagtatacagcccatggcttctcaacaaatagtacccggtaataaggttattctgccgaatccaaaagtagatgcaaaatctaccccaaacatttcagttcagaagagaagaagagtcaccagagcttgtgatgaatgtcggaaaaagaaggtcaaatgtgatggtcaacaaccatgcattcattgtaccgtttattcctatgagtgcacttacagccaaccttccagtaagaagagacagggacaatctctgagtctgagtgctccgtcaaacattaatgcaacaagttccgtacaaaaatctgtaaaacctcctgaaatcgatttccaaaggatgagagacgcactcaaatattacgaagatcttttaaaccagttgatataccccaacagtgctccaactgttcgagttaatccgattcgtctagcatcgatcttaaaacaattgagagccgataaatcaagtgatgaattaatttcagtcaaggctctttctgacaattacattgagatgcttcacaaaacgatgcaacaacctgtacagcagccagctcctccttcattggggcaaggagggtccttctctaatcacagtcccaatcataataatgcttctattgatggttccatagaatctaatctagggagggaaatacgtatcatattacctccgagagatattgcgctgaagcttatctacaagacttgggacaacgcgtgtgtacttttccgcttttatcacagacccgcatttattgaggacctgaatgagttatatgaaacagatttggcaaactacaccaataaacaacaaaggtttttacctcttgtatattcggtgatggcttgtggtgctcttttttgcaagactgatgggattaatcacggccaaaagagctccaagcccaaagactcttctgatgaaagtctcatagacgatgagggttacaagtattttattgccgcaagaaaactaatagatatcacggataccagggatacctacggaattcagactattgttatgctgatcatttttttacaatgttcggctcgtctttcaacatgctattcttatattggcattgctctaagagctgcattgagagaaggtttgcatcgtcagttgaactatcctttcaatccaattgagttagaaacaagaaagcgtcttttttggactatctataaaatggacatctatgtcaatacaatgctggggcttccaagaaccatttctgaagaggatttcgaccaggaaatgcctatcgaacttgatgatgagaacattagtgaaaccggatataggttcgatttacaaggtacaaagttatccagttcaggaatagccaatgctcacactagattgatattcataatgaagaaaattgtgaaaaaattatatcctgtcaaactacagaaaccaacctcaaacagtggcgataccccacttgagaacaatgatttattggctcatgaaatcgttcatgaacttgagatggatctccaaaattgggtcaatagtctacctgcagaactaaaaccggggatagaaccaccgaccgagtattttaaagctaacagattgcttcatttggcatacctgcatgtcaagattattctctacaggccatttattcattacatctcagaaaaggataaggttggaaatagttctatccctccgtcgcccgaagagatcacttctatcgagaaagccaagaattgtgtcaatgttgccagaattgttgttaaactagccgaagacatgattaataggaaaatgttaagtggttcatattggttttccatttataccatttttttttccgtggcatgtctggtgtactatgttcatttcgctccaccgaagaaagacaatggagaactggatccccaatacatggaaatcaagaaagatacagagagtggaagagaggtcttaaatatcctcaaagatagtagtatggcggcaagaagaacgtataatattctcaactctttgtttgagcagttaaacagaagaactgcaaaggtcaacctagcaaaggcacagcaaccaccatcagggttgaataacccagctgctacccagtatcagaaacagggtgaacacaggcagttacaaccaagtaactattctggaactgtgaaatctgtggacccagagaatatcgattactcttcctttggttctcagtttgaaaacactaacatcgaagatggttcctcaaatacaaagattgatcagaaagtgaatggggtgaactacatcgatggtgtgtttacagggatcaacctaaatatgcctaatctctcagaaacttctaacactcaaggtatcgataatccagcatttcaaagtataaacaattctaatttgaacaataattttgtacaaacaaagtacattcccggcatgatggaccagctagatatgaaaattttcggaagattccttccaccttacatgctgaactccaacaaggttgaacagggacaaaatgaaaggaacctatcaggccaaccatcctcgtcgaatactcctgatggatcacaacctgtgacagttctggatggattatacccgttgcagaatgataataataataaccacgacccaggaaattcaaagtctgttgtaaataacagtaactcggtagaaaacttactacagaactttacaatggtgccctcggggttgtcatcaacagtgcaaaatcctgaagcggcccaaaaattcaataatcatatgtcaaacatatcgaatatgaatgatccaagaagagctagcgtagctacatcagatggatccaatgacatggatcatcatagccaaggcccgataaacaaagatttgaaaccgttgagcaactacgagtttgacgatctcttctttaatgattggaccactgcgccagatacaataaattttgacagttaagtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgagatc

SEQ ID NO: 43 - DNA, 6282 bp, Artificial Sequence
  complete plasmid sequence
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgaaaccatgagcaatctacccccaacttttggttccactagacaatctccagaagaccaatcacctcccgtgcccaaggagctgtcattcaatgggaccacaccctcaggaaagctacgcttatttgtctgtcagacatgtactcgagcatttgctcgtcaggaacacttgaaacgacacgaaaggtctcacaccaaggagaaacctttcagctgcggcatttgttctcgtaaattcagccgtcgagatctgttattgagacatgcccaaaaactgcacagcaactgctctgatgcggccataacaagactaaggcgcaaggcaactcgtcggtcttctaatgccgcgggttccatatctggttctactccggtgacaacgccaaatactatgggtacgcccgaagatggcgagaaacgaaaagttcagaaactggccggccgccgggactcaaatgaacagaaactgcaactgcaacaacaacatctacagcaacaaccacagttgcaataccaacaatctcttaagcagcatgaaaatcaagtccagcagcctgatcaagatccattgatatccccgagaatgcaattattcaatgattccaaccatcacgtaaacaatttgtttgatcttggactaagaagagcttccttctccgccgttagtggaaataattatgcccattatgtgaataattttcaacaagatgcctcttctaccaatccaaatcaagattcaaataatgccgaatttgagaatattgaattttctaccccacaaatgatgcccgttgaagatgctgaaacttggatgaacaacatgggtccaattccgaacttctctctcgatgtgaacaggaacattggtgatagctttacagatatacaacacaagaattcagagcctattatatccgaaccgcccaaggacaccgctccaaacgacaagaagttgaatggctactctttttacgaagcccccatcaagccattagaatccctattttctgtcaggaatacaaagagaaacaagtataaaacaaatgacgactctccagacaccgtggataataactccgcaccggctgctaataccattcaagaacttgagtcttctttgaatgcatccaagaatttttgcttgccaactggttattccttctatggtaatttggaccaacagactttctctaacacgttatcatgcacttcttctaatgccacaatttcgcccattctactcgataactccattaataataactccactagtgacgtgagaccagaatttagaacacaaagtgtcacctctgaaatgagtcaagcccctccccctcctcaaaaaaacaactcgaaatattccaccgaagttctttttaccagcaacatgcggtcgtttattcactacgctctttccaagtatccttttattggtgtgcccactccaactcttccggagaacgaaagactaaatgaatatgctgattcattcaccaaccgtttcttaaatcattatcctttcatacatgtcacgattctcaaagaatactcccttttcaaggcaattttagatgagaatgagtcgactaagaactgggaaaataatcagttttacttagagaaccaacgaatatcaattgtttgtcttcctcttttggtggctacgataggtgcagtactatcaaacaacaaaaaggatgcttcgaatttatacgaagcttcaaggcgttgtattcatgtttacttagattccaggaaaaagatacccacttccttgtccgcaaataacaatgactctccactttggctaattcaatccctgacgttatctgttatgtatgggttatttgcggacaatgacattagtttgaatgtcgtgatcagacaagttaacgcacttaattctctggtcaagacttcgggcctgaataggacctcaattatagatcttttcaacatcaacaaacctttggataatgaactctggaatcaattcgtgaaaatagagtccaccgtaaggacaatccacacgatttttcaaatcagttccaacttaagcgccttgtacaatattattccatcgttgaaaattgatgacctaatgattactctaccagttcccacaacactttggcaagctgattcttttgtgaaattcaaaagtctaagttacggaaatcagatcccttttcaatatacaagagtactacagaatttgattgattacaatcagccattgagcgatggaaaatttttgtatgaaaaccatgtaagtgagtttggactcatatgcctacagaatggtctacaccaatacagctatttccaaaaattgactgctgtcaataacagagaagatgcgctattcacaaaggttgttaattcacttcacagttgggataggatgatttcgaattctgatttgtttccaaagaagatatatcagcagagttgcttgattttggactcaaagttgcttaataatttcctgattgtcaagagctcattgaaagtttcgaccggagacgttagttctttgaataagttaaaagaaaacgtgtggcttaaaaactggaatcaagtgtgtgctatctattataacagcttcatgaacattcctgctcccagtattcaaaagaagtacaatgacatagagtttgtggatgacatgattaatttgagtctaatcatcatcaagattatgaaactcattttctataacaatgtcaaagacaattatgaggatgaaaatgacttcaaattgcaagagttaaatttaacatttgacaattttgatgagaaaatatccttgaatttgacaatattattcgatatatttttgatgatctacaagataattaccaattacgagaagtttatgaagatcaaacacaagtttaattactacaattctaattcgaatataagcttcttgcatcatttcgaactctcctcggttatcaataacacccaaatgaaccagaatgattatatgaaaacagatattgatgaaaagcttgatcagcttttccacatctatcaaacatttttccggctgtatctggatttagaaaagtttatgaagttcaaattcaactatcatgactttgagacagagttttcaagtctctcaatatccaatatactgaacactcatgctgcttctaacaatgacacaaatgctgctgatgctatgaatgccaaggatgaaaaaatatctcccacaactttgaatagcgtattacttgctgatgaaggaaatgaaaattccggtcgtaataacgattcagaccgcctgttcatgctgaacgagctaattaattttgaagtaggtttgaaatttctcaagataggtgagtcattttttgatttcttgtatgagaataactacaagttcatccacttcaaaaacttaaatgacggaatgttccacatcaggatatacctagaaaaccgactagatggtggtgtctctagaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaagggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgagatc

SEQ ID NO: 44 - DNA, 3827 bp, Artificial Sequence
  Prm1 CDS controlled by GAP
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgaaatgcctcctaaacatcggctggagcagagtatacagcccatggcttctcaacaaatagtacccggtaataaggttattctgccgaatccaaaagtagatgcaaaatctaccccaaacatttcagttcagaagagaagaagagtcaccagagcttgtgatgaatgtcggaaaaagaaggtcaaatgtgatggtcaacaaccatgcattcattgtaccgtttattcctatgagtgcacttacagccaaccttccagtaagaagagacagggacaatctctgagtctgagtgctccgtcaaacattaatgcaacaagttccgtacaaaaatctgtaaaacctcctgaaatcgatttccaaaggatgagagacgcactcaaatattacgaagatcttttaaaccagttgatataccccaacagtgctccaactgttcgagttaatccgattcgtctagcatcgatcttaaaacaattgagagccgataaatcaagtgatgaattaatttcagtcaaggctctttctgacaattacattgagatgcttcacaaaacgatgcaacaacctgtacagcagccagctcctccttcattggggcaaggagggtccttctctaatcacagtcccaatcataataatgcttctattgatggttccatagaatctaatctagggagggaaatacgtatcatattacctccgagagatattgcgctgaagcttatctacaagacttgggacaacgcgtgtgtacttttccgcttttatcacagacccgcatttattgaggacctgaatgagttatatgaaacagatttggcaaactacaccaataaacaacaaaggtttttacctcttgtatattcggtgatggcttgtggtgctcttttttgcaagactgatgggattaatcacggccaaaagagctccaagcccaaagactcttctgatgaaagtctcatagacgatgagggttacaagtattttattgccgcaagaaaactaatagatatcacggataccagggatacctacggaattcagactattgttatgctgatcatttttttacaatgttcggctcgtctttcaacatgctattcttatattggcattgctctaagagctgcattgagagaaggtttgcatcgtcagttgaactatcctttcaatccaattgagttagaaacaagaaagcgtcttttttggactatctataaaatggacatctatgtcaatacaatgctggggcttccaagaaccatttctgaagaggatttcgaccaggaaatgcctatcgaacttgatgatgagaacattagtgaaaccggatataggttcgatttacaaggtacaaagttatccagttcaggaatagccaatgctcacactagattgatattcataatgaagaaaattgtgaaaaaattatatcctgtcaaactacagaaaccaacctcaaacagtggcgataccccacttgagaacaatgatttattggctcatgaaatcgttcatgaacttgagatggatctccaaaattgggtcaatagtctacctgcagaactaaaaccggggatagaaccaccgaccgagtattttaaagctaacagattgcttcatttggcatacctgcatgtcaagattattctctacaggccatttattcattacatctcagaaaaggataaggttggaaatagttctatccctccgtcgcccgaagagatcacttctatcgagaaagccaagaattgtgtcaatgttgccagaattgttgttaaactagccgaagacatgattaataggaaaatgttaagtggttcatattggttttccatttataccatttttttttccgtggcatgtctggtgtactatgttcatttcgctccaccgaagaaagacaatggagaactggatccccaatacatggaaatcaagaaagatacagagagtggaagagaggtcttaaatatcctcaaagatagtagtatggcggcaagaagaacgtataatattctcaactctttgtttgagcagttaaacagaagaactgcaaaggtcaacctagcaaaggcacagcaaccaccatcagggttgaataacccagctgctacccagtatcagaaacagggtgaacacaggcagttacaaccaagtaactattctggaactgtgaaatctgtggacccagagaatatcgattactcttcctttggttctcagtttgaaaacactaacatcgaagatggttcctcaaatacaaagattgatcagaaagtgaatggggtgaactacatcgatggtgtgtttacagggatcaacctaaatatgcctaatctctcagaaacttctaacactcaaggtatcgataatccagcatttcaaagtataaacaattctaatttgaacaataattttgtacaaacaaagtacattcccggcatgatggaccagctagatatgaaaattttcggaagattccttccaccttacatgctgaactccaacaaggttgaacagggacaaaatgaaaggaacctatcaggccaaccatcctcgtcgaatactcctgatggatcacaacctgtgacagttctggatggattatacccgttgcagaatgataataataataaccacgacccaggaaattcaaagtctgttgtaaataacagtaactcggtagaaaacttactacagaactttacaatggtgccctcggggttgtcatcaacagtgcaaaatcctgaagcggcccaaaaattcaataatcatatgtcaaacatatcgaatatgaatgatccaagaagagctagcgtagctacatcagatggatccaatgacatggatcatcatagccaaggcccgataaacaaagatttgaaaccgttgagcaactacgagtttgacgatctcttctttaatgattggaccactgcgccagatacaataaattttgacagttaagtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcc

SEQ ID NO: 45 - DNA, 4368 bp, Artificial Sequence
  Mxr CDS controlled by GAP
agatcttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatttcgaaaccatgagcaatctacccccaacttttggttccactagacaatctccagaagaccaatcacctcccgtgcccaaggagctgtcattcaatgggaccacaccctcaggaaagctacgcttatttgtctgtcagacatgtactcgagcatttgctcgtcaggaacacttgaaacgacacgaaaggtctcacaccaaggagaaacctttcagctgcggcatttgttctcgtaaattcagccgtcgagatctgttattgagacatgcccaaaaactgcacagcaactgctctgatgcggccataacaagactaaggcgcaaggcaactcgtcggtcttctaatgccgcgggttccatatctggttctactccggtgacaacgccaaatactatgggtacgcccgaagatggcgagaaacgaaaagttcagaaactggccggccgccgggactcaaatgaacagaaactgcaactgcaacaacaacatctacagcaacaaccacagttgcaataccaacaatctcttaagcagcatgaaaatcaagtccagcagcctgatcaagatccattgatatccccgagaatgcaattattcaatgattccaaccatcacgtaaacaatttgtttgatcttggactaagaagagcttccttctccgccgttagtggaaataattatgcccattatgtgaataattttcaacaagatgcctcttctaccaatccaaatcaagattcaaataatgccgaatttgagaatattgaattttctaccccacaaatgatgcccgttgaagatgctgaaacttggatgaacaacatgggtccaattccgaacttctctctcgatgtgaacaggaacattggtgatagctttacagatatacaacacaagaattcagagcctattatatccgaaccgcccaaggacaccgctccaaacgacaagaagttgaatggctactctttttacgaagcccccatcaagccattagaatccctattttctgtcaggaatacaaagagaaacaagtataaaacaaatgacgactctccagacaccgtggataataactccgcaccggctgctaataccattcaagaacttgagtcttctttgaatgcatccaagaatttttgcttgccaactggttattccttctatggtaatttggaccaacagactttctctaacacgttatcatgcacttcttctaatgccacaatttcgcccattctactcgataactccattaataataactccactagtgacgtgagaccagaatttagaacacaaagtgtcacctctgaaatgagtcaagcccctccccctcctcaaaaaaacaactcgaaatattccaccgaagttctttttaccagcaacatgcggtcgtttattcactacgctctttccaagtatccttttattggtgtgcccactccaactcttccggagaacgaaagactaaatgaatatgctgattcattcaccaaccgtttcttaaatcattatcctttcatacatgtcacgattctcaaagaatactcccttttcaaggcaattttagatgagaatgagtcgactaagaactgggaaaataatcagttttacttagagaaccaacgaatatcaattgtttgtcttcctcttttggtggctacgataggtgcagtactatcaaacaacaaaaaggatgcttcgaatttatacgaagcttcaaggcgttgtattcatgtttacttagattccaggaaaaagatacccacttccttgtccgcaaataacaatgactctccactttggctaattcaatccctgacgttatctgttatgtatgggttatttgcggacaatgacattagtttgaatgtcgtgatcagacaagttaacgcacttaattctctggtcaagacttcgggcctgaataggacctcaattatagatcttttcaacatcaacaaacctttggataatgaactctggaatcaattcgtgaaaatagagtccaccgtaaggacaatccacacgatttttcaaatcagttccaacttaagcgccttgtacaatattattccatcgttgaaaattgatgacctaatgattactctaccagttcccacaacactttggcaagctgattcttttgtgaaattcaaaagtctaagttacggaaatcagatcccttttcaatatacaagagtactacagaatttgattgattacaatcagccattgagcgatggaaaatttttgtatgaaaaccatgtaagtgagtttggactcatatgcctacagaatggtctacaccaatacagctatttccaaaaattgactgctgtcaataacagagaagatgcgctattcacaaaggttgttaattcacttcacagttgggataggatgatttcgaattctgatttgtttccaaagaagatatatcagcagagttgcttgattttggactcaaagttgcttaataatttcctgattgtcaagagctcattgaaagtttcgaccggagacgttagttctttgaataagttaaaagaaaacgtgtggcttaaaaactggaatcaagtgtgtgctatctattataacagcttcatgaacattcctgctcccagtattcaaaagaagtacaatgacatagagtttgtggatgacatgattaatttgagtctaatcatcatcaagattatgaaactcattttctataacaatgtcaaagacaattatgaggatgaaaatgacttcaaattgcaagagttaaatttaacatttgacaattttgatgagaaaatatccttgaatttgacaatattattcgatatatttttgatgatctacaagataattaccaattacgagaagtttatgaagatcaaacacaagtttaattactacaattctaattcgaatataagcttcttgcatcatttcgaactctcctcggttatcaataacacccaaatgaaccagaatgattatatgaaaacagatattgatgaaaagcttgatcagcttttccacatctatcaaacatttttccggctgtatctggatttagaaaagtttatgaagttcaaattcaactatcatgactttgagacagagttttcaagtctctcaatatccaatatactgaacactcatgctgcttctaacaatgacacaaatgctgctgatgctatgaatgccaaggatgaaaaaatatctcccacaactttgaatagcgtattacttgctgatgaaggaaatgaaaattccggtcgtaataacgattcagaccgcctgttcatgctgaacgagctaattaattttgaagtaggtttgaaatttctcaagataggtgagtcattttttgatttcttgtatgagaataactacaagttcatccacttcaaaaacttaaatgacggaatgttccacatcaggatatacctagaaaaccgactagatggtggtgtctctagaacaaaaactcatctcagaagaggatctgaatagcgccgtcgaccatcatcatcatcatcattgagttttagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcc